(19) United States
(12) Patent Application Publication
(10) Pub. No.: US 2023/0364005 A1
(43) Pub. Date: Nov. 16, 2023

(54) Sunscreen Compositions and Methods of Use

(71) Applicant: Colgate-Palmolive Company, New York, NY (US)

(72) Inventor: Devon Sean COCUZZA, Flemington, NJ (US)

(73) Assignee: Colgate-Palmolive Company, New York, NY (US)

(21) Appl. No.: 18/318,100

(22) Filed: May 16, 2023

Related U.S. Application Data
(60) Provisional application No. 63/342,394, filed on May 16, 2022.

(51) Int. Cl. A61K 8/9789; A61K 8/29; A61K 8/27; A61K 8/37; A61Q 17/00

(52) U.S. Cl. CPC A61K 8/9789; A61K 8/29; A61K 8/27; A61K 8/37; A61Q 17/00

(57) ABSTRACT

The present disclosure relates to broad spectrum sunscreen compositions, and to methods for using broad spectrum sunscreen compositions to protect the skin from UV radiation. In at least one aspect the sunscreen comprises: a metal oxide (e.g., zinc oxide) one or more SPF active ingredients (e.g., octinoxate and/or octisalate) and one or more SPF enhancement ingredients (e.g., a photo stabilizer (e.g., butyloctyl salicylate and/or ethylhexyl methoxycrylene) and/or an antioxidant (e.g., a plant extract) (e.g., pongamia pinnata extract).

### CROSS-REFERENCE TO RELATED APPLICATIONS

This application claims the benefit of priority from U.S. Provisional Application No. 63/342,394, filed May 16, 2022, entitled “Sunscreen Compositions and Methods of Use,” the contents of which are hereby incorporated herein in their entireties, for all purposes.

### FIELD OF THE DISCLOSURE

The present disclosure relates to broad spectrum sunscreen compositions, and to methods for using broad spectrum sunscreen compositions to protect the skin from UV radiation.

### BACKGROUND

The negative effects of exposure to ultraviolet (“UV”) light are well known. Prolonged exposure to sunlight causes damage such as sunburn to the skin and hair. Exposure to UV light at certain wavelengths, from about 290 nm to about 400 nm, for example, may lead to long term damage as well as serious conditions such as skin cancer.

For example, ultraviolet radiation in the shorter wavelength UVB region of 290 to 320 nm is considered significant in causing burning. Accordingly, protection against UVB radiation has traditionally been a primary target of sunscreen formulations. Relatively recently there has also been a desire for further protection against UVA radiation to reduce long term skin deterioration. Generally, UV light contributes to aging by causing free radicals, such as O⁻, OH, superoxide anion, nitric oxide, and hydrogen radicals, to form in the skin. Free radicals attack DNA, membrane lipids, and proteins, generating carbon radicals. These in turn react with oxygen to produce a peroxyl radical that can attack adjacent fatty acids to generate new carbon radicals. The end result is a loss of skin elasticity and the appearance of wrinkles.

More importantly, both the UVA and UVB wavebands have been identified as potential factors for increasing skin cancers, e.g., melanomas. Skin cancer is one of the most common cancer types and the incidence in the US is problematic. For these and other reasons, consistent use of a topical sunscreen is now strongly recommended and is widespread.

Sunscreens are subject to strict regulatory approval by various federal agencies in order to be considered “broad spectrum.” For example, to be considered a “broad spectrum” sunscreen, the current federal standard (21 C.F.R. § 201.327) requires that the mean critical wavelength must be 370 nm or greater. The critical wavelength is identified as the wavelength at which the integral of the spectral absorbance curve reaches 90 percent of the integral over the UV spectrum from 290 to 400 nm. In addition to this requirement, there are regulatory proposals that would require “broad spectrum” sunscreens to meet a UVAI/UV ratio requirement of greater than or equal to 0.70. Accordingly, there is an unmet need for broad spectrum sunscreens that can provide adequate protection from UV light and also meet the changing regulatory standards.

### BRIEF SUMMARY

The disclosure provides novel sunscreens that are believed to unexpectedly provide a UVAI/UV ratio requirement of greater than or equal to 0.70 while simultaneously maintaining a sun protection factor (“SPF”) of 40 and above.

Achieving a desirable UVAI/UV ratio while maintaining a relatively high SPF (e.g., ≥40) is difficult because increasing or maintaining a high SPF increases or maintains a large denominator, which results in a lower UVAI/UV ratio. The formulation for deriving the UVAI/UV ratio is as follows:

UVAI/UV=average absorbance 340 nm−400 nm/average absorbance UVB(290 nm−320 nm)+UVAII(320 nm−340 nm)+UVAI(340 nm−400 nm).

In view of the above formulation for deriving the UVAI/UV ratio, one of skill would likely recognize that reducing the UVB coverage would increase the ratio, e.g., by reducing the UVB coverage this simultaneously reduces the denominator of the above equation and therefore increase the overall ratio. However, approximately 90% of the erythemal contribution from the sun is contributed from wavelengths in the range of 290 nm-320 nm (UVB). And since SPF is a measure of a sunscreen's ability to reduce erythema, it is effectively a measure of the ability of a product to block UVB light (290 nm-320 nm). Therefore, one of the simplest and most direct routes to improving the UVAI/UV ratio would be to reduce the UVB coverage, which would have the undesirable effect of simultaneously reducing the SPF value of a sunscreen.

The sunscreen compositions of the instant disclosure surprisingly achieve both a UVAI/UV ratio of greater than or equal to 0.70 and a SPF value of 40 and more. Furthermore, the disclosed sunscreen compositions can possess a UVAI/UV ratio of greater than or equal to 0.70 and a SPF value of 40 or more while achieving commercially desirable aesthetics. Accordingly, in one aspect, the sunscreen compositions described herein comprise a metal oxide (e.g., zinc oxide), one or more SPF active ingredients (e.g., octinoxate and/or octisalate) and one or more SPF enhancement ingredients (e.g., a photostabilizer (e.g., butyloctyl salicylate and/or ethylhexyl methoxycrylene) and/or an antioxidant (e.g., a plant extract) (e.g., pongamia pinnata extract)). In a further aspect the resulting sunscreen comprises a metal oxide (e.g., zinc oxide), one or more SPF active ingredients (e.g., octinoxate and/or octisalate) and one or more SPF enhancement ingredients (e.g., a photostabilizer (e.g., butyloctyl salicylate and/or ethylhexyl methoxycrylene) and/or an antioxidant (e.g., a plant extract) (e.g., pongamia pinnata extract) and has a UVAI/UV ratio requirement of greater than or equal to 0.70 (e.g., from 0.7-0.8) and an SPF that is 40 and above.

### DETAILED DESCRIPTION

The following description of the preferred embodiment(s) is merely exemplary in nature and is in no way intended to limit the disclosure, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight of the entire composition. The amounts given are based on the active weight of the material unless specified otherwise.

In one aspect the disclosure provides a sunscreen composition (e.g., Composition 1.0) wherein the sunscreen composition comprises:
a metal oxide (e.g., zinc oxide)
one or more SPF active ingredients (e.g., octinoxate and/or octisalate) and
one or more SPF enhancement ingredients (e.g., a photostabilizer (e.g., butyloctyl salicylate and/or ethylhexyl methoxycrylene) and/or an antioxidant (e.g., a plant extract) (e.g., pongamia pinnata extract); and
wherein the UVAI/UV ratio of the sunscreen composition is greater than or equal to 70 and the SPF is greater than or equal to 40.

For example, the sunscreen compositions of the present disclosure may include:
1.1 Composition 1.0, wherein the metal oxide is selected from the group consisting of: Ba, Zn, Ti, V, Cr, Mn, Fe, Co, Ni, Cu, Re, Os, Jr, and Sn. Non-limiting examples of metal oxides include titanium oxide (amorphous or crystallized in rutile and/or anatase form), iron oxide, zinc oxide, zirconium oxide, and cerium oxide. The metal oxides may comprise treated or untreated metal oxides. Treated metal oxides typically have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminium salts of fatty acids, metal (titanium or aluminium) alkoxides, polyethylene, silicones, proteins (collagen or elastin), alkanolamines, silicon oxides, metal oxides, sodium hexametaphosphate, alumina, or glycerol. The metal oxide may have a mean particle size of about 5 nm to about 30 μm. In some instances, the mean particle size of the metal oxide may be about 5 nm to about 25 μm, about 10 nm to about 10 μm, or about 15 nm to about 5 μm. In further instances, the metal oxide may a mean particle size of about 5 nm to about 100 nm, about 5 nm to about 75 nm, or about 10 nm to 50 nm. Larger particles sizes may also be useful, such as, e.g., from about 1 μm to about 25 μm, about 5 μm to about 20 μm, or about 10 μm to about 15 μm. The treatment metal oxides may having a coating formed thereon. In certain embodiments, the sunscreen composition may include treated and/or coated metal oxides in an amount from about 0.01 to about 3 wt. %, based on the total weight of the sunscreen composition. For instance, the amount of treated and/or coated metal oxides in the sunscreen composition may be from about 0.01 to about 3 wt. %, about 0.01 to about 2 wt. %, about 0.01 to about 1 wt. %, about 0.01 to about 0.8 wt. %, about 0.01 to about 0.6 wt. %, about 0.01 to about 0.4 wt. %, about 0.01 to about 0.2 wt. %, about 0.01 to about 0.1 wt. %; from about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.2 to about 1 wt. %, about 0.2 to about 0.8 wt. %, about 0.2 to about 0.6 wt. %, about 0.2 to about 0.4 wt. %; from about 0.5 to about 1 wt. %, about 0.5 to about 0.8 wt. %, about 0.5 to about 0.6 wt. %; from about 0.8 to about 1 wt. %, about 0.8 to about 1.5 wt. %, about 0.8 to about 2 wt. %, or any range or subrange thereof, based on the total weight of the sunscreen composition. In certain embodiments, the amount of treated and/or coated metal oxides in the sunscreen composition is about 0.01 wt. %, about 0.05 wt. %, about 0.1 wt. %, about 0.15 wt. %, about 0.2 wt. %, or any range or subrange thereof, based on the total weight of the sunscreen composition.
1.2 Composition 1.1, wherein the metal oxide comprises zinc oxide and/or titanium oxide. The zinc oxide may be selected from treated and/or coated zinc oxide. Additionally or alternatively, the titanium oxide may be selected from treated and/or coated titanium oxide. Examples of treated and/or coated zinc oxides include zinc oxides coated with silica and polymethylhydrogenosiloxane, zinc oxides coated with polymethylhydrogenosiloxane, zinc oxides coated with perfluoroalkyl phosphate and copolymer based on perfluoroalkylethyl as a dispersion in cyclopentasiloxane, and zinc oxides coated with silica and polymethylsilsesquioxane. Examples of treated and/or coated titanium oxides include those treated with: silica and alumina; alumina and aluminium stearate; iron oxides and iron stearate; silica, alumina and silicone; sodium hexametaphosphate; octyltrimethoxysilane; alumina and stearic acid; alumina and glycerol; alumina and silicone; and combinations thereof.
1.3 Any of composition 1.0-1.2, wherein the metal oxide is present in the composition from about 5 to about 25 wt. % or from 5 to about 15 wt. %, relative to the total weight of the sunscreen composition (e.g., from about 7.5% by wt.-about 12% by wt.). For example, the metal oxide may be present in the sunscreen composition in an amount from about 5 to about 25 wt. %, about 7 to about 25 wt. %, about 9 to about 25 wt. %, about 11 to about 25 wt. %, about 13 to about 25 wt. %, about 15 to about 25 wt. %, about 18 to about 25 wt. %; from about 5 to about 21 wt. %, about 7 to about 21 wt. %, about 9 to about 21 wt. %, about 11 to about 21 wt. %, about 13 to about 21 wt. %, about 15 to about 21 wt. %, about 18 to about 21 wt. %; from about 5 to about 18 wt. %, about 7 to about 18 wt. %, about 9 to about 15 wt. %, about 11 to about 18 wt. %, about 13 to about 18 wt. %, about 15 to about 18 wt. %; from about 5 to about 15 wt. %, about 7 to about 15 wt. %, about 9 to about 15 wt. %, about 11 to about 15 wt. %, about 13 to about 15 wt. %; from about 5 to about 13 wt. %, about 7 to about 13 wt. %, about 9 to about 13 wt. %, about 11 to about 13 wt. %; from about 5 to about 11 wt. %, about 7 to about 11 wt. %, about 9 to about 11 wt. %; from about 5 to about 9 wt. %, about 7 to about 9 wt. %; from about 5 to about 8 wt. %, about 7 to about 8 wt. %, about 5 to about 7 wt. %, or any range or subrange thereof, based on the total weight of the sunscreen composition.
1.4 Composition 1.3, wherein the metal oxide comprises zinc oxide in an amount from 5 to 25 wt. %, relative to the total weight of the sunscreen composition (e.g., from about 7.5% by wt.-about 12% by wt.) (e.g., about 7% by wt.) (e.g., about 8% by wt.) (e.g., about 9% by wt.) (e.g., about 10% by wt.) (e.g., about 11% by wt.).
1.5 Any of the preceding compositions, wherein the one or more SPF active ingredients is selected from the group consisting of: octinoxate, octisalate, homosalate, avobenzone, octocrylene, para-aminobenzoic acid (PABA), cinoxate, dioxybenzone, menthyl anthranilate, padimate O, ensulizole, sulisobenzone, trolamine salicylate, ecamsule, 4-methylbenzylidene camphor, diethylamino hydroxybenzoyl hexyl benzoate, bis diethylamino hydroxybenzoyl benzoate, disodium phenyl dibenzimidazole tetrasulfonate, Parsol SLX (INCI Polysilicone-15), Butyl Methoxydibenzoylmethane, Ethylhexyl Methoxycinnamate, Ethylhexyl Salicylate, Benzophenone-3, Ethylhexyl Dimethyl PABA, Phenylbenzimidazole Sulfonic Acid, Benzophenone-4, Tris-biphenyl triazine, Bis-Ethylhexyloxyphenol Methoxyphenyl Triazine, Methylene Bis-Benzotriazolyl Tetramethylbutylphenol, Ethylhexyl triazone, Methoxypropylamino Cyclohexenylidene Ethoxyethylcyanoacetate, Phenylene Bis-Diphenyltriazine, Isoamyl p-Methoxycinnamate, Benzylidene Camphor Sulfonic Acid, Camphor benzalkonium methosulfate, Diethylamino Hydroxybenzoyl Hexyl Benzoate, Disodium Phenyl Dibenzimidazole Tetrasulfonate, Drometrizole Trisiloxane, PEG-25 PABA, Polyacrylamidomethyl Benzylidene Camphor, Benzophenone-5, benzophenone-8, benzophenone-8, Diethylhexyl butamido triazone, and a combination of two or more thereof. The SPF active ingredients may be selected from certain UV filters, such as organic UV filters. Examples of organic UV filters include ethylhexyl triazone, bis-ethylhexyloxyphenol methoxyphenyltriazine, dioctyl butamido triazone, drometrizole trisiloxane, and isopentyl 4-methoxycinnamate.
1.6 Composition 1.5, wherein the SPF active ingredient comprises octinoxate and/or octisalate.
1.7 Any of the preceding compositions, wherein the SPF active ingredient is present in the composition from 1 to 10 wt. %, relative to the total weight of the sunscreen composition. In some embodiments, the sun care composition comprises one or more SPF active ingredient(s) in an amount from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %; from about 7 to about 10 wt. %, about 7 to about 8 wt. %, or any range or subrange thereof, based on the total weight of the sunscreen composition.
1.8 Any of the preceding sunscreen compositions wherein the one or more SPF enhancement ingredients comprises one or more photostabilizer(s) and/or an antioxidant.
1.9 Any of the preceding sunscreen compositions, wherein the sunscreen composition includes a photostabilizer selected from the group consisting of: ethylhexyl triazone, dioctyl butamido triazone, benzylidene malonate polysiloxane, bis benzoxazoylphenyl ethylhexylimino triazine, drometrizole trisiloxane, methylene bis-benzotriazolyl tetramethylbutylphenol, bis-ethylhexyloxyphenol methoxyphenyltriazine, butyloctyl salicylate, alpha-cyanodiphenylacrylate (e.g., ethylhexyl methoxycrylene or 2-ethylhexyl 2-cyano-3-(4-methoxyphenyl)-3-phenylpropenoate), isopentyl 4-methoxycinnamate, diethylhexyl syringylidenemalonate, diethylhexyl 2,6-naphthalate, octocrylene and combinations thereof. In some embodiments, the photostabilizer comprises dioctyl butamido triazone, benzylidene malonate polysiloxane, butyloctyl salicylate, alpha-cyanodiphenylacrylate (e.g., ethylhexyl methoxycrylene or 2-ethylhexyl 2-cyano-3-(4-methoxyphenyl)-3-phenylpropenoate), isopentyl 4-methoxycinnamate, diethylhexyl syringylidenemalonate, diethylhexyl 2,6-naphthalate, octocrylene, or a combination of two or more thereof.
1.10 The sunscreen composition of 1.9, wherein the photostabilizer comprises butyloctyl salicylate and/or ethylhexyl methoxycrylene.
1.11 Any of the preceding sunscreen compositions, wherein the photostabilizer is present in the sunscreen composition in an amount from 0.5 to about 10 wt. %, relative to the total weight of the sunscreen composition (e.g., from 1% by wt.-7.5% by wt.) (e.g., from 2% by wt.-5% by wt.) (e.g., about 2% by wt.) (e.g., about 3% by wt.) (e.g., about 3.5% by wt.) (e.g., about 4% by wt.) (e.g., about 4.5% by wt.) (e.g., about 5% by wt.). For instance, the sunscreen composition may comprise the photostabilizer in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %; from about 7 to about 10 wt. %, about 7 to about 8 wt. %, or any range or subrange thereof, based on the total weight of the sunscreen composition
1.12 Any of the preceding sunscreen compositions, wherein the photostabilizer comprises butyloctyl salicylate from about 0.5 to about 10 wt. %, relative to the total weight of the sunscreen composition (e.g., from 1% by wt.-7.5% by wt.) (e.g., from 2% by wt.-5% by wt.) (e.g., about 2% by wt.) (e.g., about 3% by wt.) (e.g., about 3.5% by wt.) (e.g., about 4% by wt.) (e.g., about 4.5% by wt.) (e.g., about 5% by wt.). In some embodiments, the butyloctyl salicylate is present in the sunscreen composition in any of the above amounts listed for the photostabilizer.
1.13 Any of the preceding sunscreen compositions, wherein the photostabilizer comprises ethylhexyl methoxycrylene from about 0.5 to about 10 wt. %, relative to the total weight of the sunscreen composition (e.g., from 1% by wt.-7.5% by wt.) (e.g., from 2% by wt.-5% by wt.) (e.g., about 2% by wt.) (e.g., about 3% by wt.) (e.g., about 3.5% by wt.) (e.g., about 4% by wt.) (e.g., about 4.5% by wt.) (e.g., about 5% by wt.). In some embodiments, the ethylhexyl methoxycrylene is present in the sunscreen composition in any of the above amounts listed for the photostabilizer.
1.14 Any of the preceding sunscreen compositions wherein the SPF enhancement ingredient comprises an antioxidant.
1.15 The sunscreen composition of 1.14, wherein the antioxidant is selected from Angelica archangelica, Arnica montana, Camellia sinensis, Cupressus server, Coffee arabica, Polygonatum multiflorum, Pongamia pinnata extract, Rosmarinus officinalis, Evemia fufuracea, Evernia prunastri, Aventa sativa, vitamin A, vitamin E, peptides, flavones, flavonols, and a combination of two or more thereof.
1.16 Any of the preceding sunscreen compositions, such as the sunscreen composition of 1.15, wherein the antioxidant is present in an amount from about 0.1 to about 5 wt. %, relative to the total weight of the sunscreen composition (e.g., from 0.1% by wt.-1% by wt.) (e.g., from 0.1% by wt.-0.5% by wt.) (e.g., about 0.1% by wt.) (e.g., about 0.2% by wt.) (e.g., about 0.25% by wt.) (e.g., about 0.3% by wt.) (e.g., about 0.4% by wt.) (e.g., about 0.5% by wt.). In certain instances, the sunscreen composition includes one or more antioxidant(s) in an amount from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, about 4 to about 5 wt. %, or any range or subrange thereof, based on the total weight of the sunscreen composition. The total amount of antioxidant(s) in the oral care composition may in certain embodiments be from about 0.01 to about 1 wt. %, about 0.01 to about 0.8 wt. %, about 0.01 to about 0.5 wt. %, about 0.01 to about 0.3 wt. %, about 0.01 to about 0.2 wt. %, about 0.01 to about 0.1 wt. %; from about 0.05 to about 1 wt. %, about 0.05 to about 0.8 wt. %, about 0.05 to about 0.5 wt. %, about 0.05 to about 0.3 wt. %, about 0.05 to about 0.2 wt. %, about 0.05 to about 0.1 wt. %; from about 0.1 to about 1 wt. %, about 0.1 to about 0.8 wt. %, about 0.1 to about 0.5 wt. %, about 0.1 to about 0.3 wt. %, about 0.1 to about 0.2 wt. %, about 0.1 to about 0.1 wt. %, or any range or subrange thereof, based on the total weight of the sunscreen composition.
1.17 The sunscreen composition of 1.15 or 1.16, wherein the antioxidant comprises pongamia pinnata extract, and wherein the pongamia pinnata extract is selected from: Pongamia Glabra Root Extract, Pongamia Glabra Seed Oil, Pongamia Glabra Seed Oil C16-24 Isoalkyl Esters, Pongamia Pinnata Fruit Extract, Pongamia Pinnata Leaf Extract, and Pongamia Pinnata Seed Extract.
1.18 The sunscreen composition of 1.17, wherein the pongamia pinnata extract is a pongamia pinnata seed extract, and wherein the pongamia pinnata seed extract is present in an amount from about 0.01 to about 5 wt. % or about 0.1 to about 5 wt. %, relative to the total weight of the sunscreen composition (e.g., from about 0.01 to 1 wt. %) (e.g., from about 0.05 to about 0.5 wt. %) (e.g., about 0.05 to about 0.2 wt. %) (e.g., about 0.05 to about 0.1 wt. %) (e.g., about 0.2 wt. %) (e.g., about 0.25 wt. %) (e.g., about 0.3 wt. %) (e.g., about 0.4 wt. %) (e.g., about 0.5 wt. %). For instance, the sunscreen composition may comprise pongamia pinnata extract in an amount from about 0.01 to about 1 wt. %, about 0.01 to about 0.8 wt. %, about 0.01 to about 0.5 wt. %, about 0.01 to about 0.3 wt. %, about 0.01 to about 0.2 wt. %, about 0.01 to about 0.1 wt. %; from about 0.05 to about 1 wt. %, about 0.05 to about 0.8 wt. %, about 0.05 to about 0.5 wt. %, about 0.05 to about 0.3 wt. %, about 0.05 to about 0.2 wt. %, about 0.05 to about 0.1 wt. %; from about 0.1 to about 1 wt. %, about 0.1 to about 0.8 wt. %, about 0.1 to about 0.5 wt. %, about 0.1 to about 0.3 wt. %, about 0.1 to about 0.2 wt. %, about 0.1 to about 0.1 wt. %, or any range or subrange thereof, based on the total weight of the sunscreen composition. In some embodiments, the pongamia pinnata seed extract is present in the sunscreen composition in any of the above amounts listed for the antioxidant.
1.19 The sunscreen composition of any of the preceding sunscreen compositions, wherein the sunscreen composition comprises:
   zinc oxide;
   octinoxate and/or octisalate; and
   butyloctyl salicylate, ethylhexyl methoxycrylene, pongamia pinnata extract,
      wherein the UVAI/UV ratio of the sunscreen is greater than or equal to 70 and the SPF is greater than or equal to 40.
1.20 The sunscreen composition of any of the preceding sunscreen compositions, wherein the sunscreen composition comprises:
   zinc oxide in an amount from 5 to 15 wt. % (e.g., about 9% by wt.) (e.g., about 11% by wt.);
   octisalate in an amount from 1 to 10 wt. % (e.g., about 4% by wt.);
   butyloctyl salicylate in an amount from 0.5 to 5 wt. % (e.g., about 2% by wt.);
   ethylhexyl methoxycrylene in an amount from 0.5 to 5 wt. % (e.g., about 2% by wt.);
   pongamia pinnata extract in an amount from 0.1 to 1 wt. % (e.g., about 0.25% by wt.); and
      wherein the UVAI/UV ratio of the sunscreen is greater than or equal to 70 and the SPF is greater than or equal to 40, and wherein all weight percentages are based on the total weight of the sunscreen composition.
1.21 The composition of any of the preceding sunscreen compositions, wherein the sunscreen composition comprises:
   zinc oxide in an amount from about 5 to about 15 wt. % (e.g., about 9% by wt.) (e.g., about 11% by wt.);
   octisalate in an amount from about 1 to about 10 wt. % (e.g., about 4% by wt.);
   butyloctyl salicylate in an amount from about 0.5 to about 5 wt. % (e.g., about 2% by wt.);
   ethylhexyl methoxycrylene in an amount from about 0.5 to about 5 wt. % (e.g., about 2% by wt.);
   pongamia pinnata extract in an amount from about 0.1 to about 1 wt. (e.g., about 0.25% by wt.); and
   optionally, titanium dioxide in an amount from about 0.1 to 2 wt. % (e.g., about 1% by wt.),
      wherein the UVAI/UV ratio of the sunscreen is greater than or equal to 70 and the SPF is greater than or equal to 40 and wherein all weight percentages are based on the total weight of the sunscreen composition.
1.22 The composition of any of the preceding sunscreen compositions, wherein the sunscreen composition comprises:
   zinc oxide in an amount from about 5 to about 15 wt. % (e.g., about 9% by wt.) (e.g., about 11% by wt.);
   octinoxate in an amount from about 1 to about 10 wt. % (e.g., about 2% by wt.) (e.g., about 3.5% by wt.) (e.g., about 4% by wt.);
   butyloctyl salicylate in an amount from about 0.5 to about 5 wt. % (e.g., about 2% by wt.);
   ethylhexyl methoxycrylene from about 0.5 to about 5 wt. % (e.g., about 2% by wt.); and
   pongamia pinnata extract in an amount from 0.1 to 1 wt. % (e.g., about 0.25% by wt.),
      wherein the UVAI/UV ratio of the sunscreen is greater than or equal to 70 and the SPF is greater than or equal to 40 and wherein all weight percentages are based on the total weight of the sunscreen composition.
1.23 Any of the preceding sunscreen compositions, wherein the sunscreen composition further comprises water (e.g., 50%-70% by wt.) (e.g., about 62% by wt.). The sunscreen compositions may include water in an amount from about 50 to about 70 wt. %, about 50 to about 67 wt. %, about 50 to about 64 wt. %, about 50 to about 62 wt. %, about 50 to about 60 wt. %, about 50 to about 57 wt. %; from about 53 to about 70 wt. %, about 53 to about 67 wt. %, about 53 to about 64 wt. %, about 53 to about 62 wt. %, about 53 to about 60 wt. %, about 53 to about 57 wt. %; from about 56 to about 70 wt. %, about 56 to about 67 wt. %, about 56 to about 64 wt. %, about 56 to about 62 wt. %, about 56 to about 60 wt. %; from about 59 to about 70 wt. %, about 59 to about 67 wt. %, about 59 to about 64 wt. %, about 59 to about 62 wt. %; from about 61 to about 70 wt. %, about 61 to about 67 wt. %, about 61 to about 64 wt. %, about 61 to about 62 wt. %; from about 63 to about 70 wt. %, about 63 to about 67 wt. %, or any range or subrange thereof, based on the total weight of the sunscreen composition.
1.24 Any of the preceding sunscreen compositions, wherein the sunscreen composition is in the form of a cream, a gel, a paste, a lotion, a rinse, a foam, an emulsion, a spray, and combinations thereof.
1.25 Any of the preceding sunscreen compositions for use in a cosmetic, dermatologic, pharmaceutical or medicament composition.
1.26 Any of the preceding sunscreen compositions, wherein the composition is applied topically.
1.27 Any of the preceding sunscreen compositions further comprising one or more ingredients selected from: self-tanning agents, free-radical scavengers, vitamins, enzymes, herbal ingredients, polymers, antioxidants, omega-3/6 fatty acids, and a combination of two or more thereof.
1.28 Any of the preceding skin care compositions, wherein the sunscreen composition comprises an additional active ingredient selected from: carnosine, cica (asiaticoside), Vitamin E (e.g., alpha-tocopherol, gamma-tocopherol, beta-tocopherol, delta-tocopherol, and combinations thereof) hyaluronic acid and salts thereof (e.g., sodium hyaluronate), magnesium sulfate, saccharide isomerate, a keto-carotenoid (e.g., astaxanthin), bisabolol, Vitamin B (e.g., niacinamide), nicotinamide adenine dinucleotide (NAD), ginger extract, and a combination of two or more thereof.
1.29 Any of the preceding sunscreen compositions, wherein the sunscreen composition comprises:
   a metal oxide from about 5 to about 25 wt. %, relative to the total weight of the sunscreen composition (e.g., from 7 to 13 wt. %);
   one or more SPF active ingredients is selected from the group consisting of octinoxate, octisalate, homosalate, avobenzone, octocrylene, para-aminobenzoic acid, cinoxate, dioxybenzone, menthyl anthranilate, padimate 0, ensulizole, sulisobenzone, trolamine salicylate, ecamsule, 4-methylbenzylidene camphor, diethylamino hydroxybenzoyl hexyl benzoate, bis diethylamino hydroxybenzoyl benzoate, disodium phenyl dibenzimidazole tetrasulfonate, Parsol SLX (INCI Polysilicone-15), Butyl Methoxydibenzoylmethane, Ethylhexyl Methoxycinnamate, Ethylhexyl Salicylate, Benzophenone-3, Ethylhexyl Dimethyl PAB A, Phenylbenzimidazole Sulfonic Acid, Benzophenone-4, Tris-biphenyl triazine, Bis-Ethylhexyloxyphenol Methoxyphenyl Triazine, Methylene Bis-Benzotriazolyl Tetramethylbutylphenol, Ethylhexyl triazone, Methoxypropylamino Cyclohexenylidene Ethoxyethylcyanoacetate, Phenylene Bis-Diphenyltriazine, Isoamyl p-Methoxycinnamate, Benzylidene Camphor Sulfonic Acid, Camphor benzalkonium methosulfate, Diethylamino Hydroxybenzoyl Hexyl Benzoate, Disodium Phenyl Dibenzimidazole Tetrasulfonate, Drometrizole Trisiloxane, PEG-25 PABA, Polyacrylamidomethyl Benzylidene Camphor, Benzophenone-5, benzophenone-8, Diethylhexyl butamido triazone, and a combination of two or more thereof.
   one or more SPF enhancement ingredients comprising a photostabilizer and/or an antioxidant,
   wherein the photostabilizer is selected from the group consisting of:
ethylhexyl triazone, dioctyl butamido triazone, benzylidene malonate polysiloxane, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, butyloctyl salicylate, an alpha-cyanodiphenylacrylate (e.g., ethylhexyl methoxycrylene or 2-ethylhexyl 2-cyano-3-(4-methoxyphenyl)-3-phenylpropenoate), isopentyl 4-methoxycinnamate, and combinations thereof; and
   wherein the UVAI/UV ratio of the sunscreen is greater than or equal to 70 and the SPF is greater than or equal to 40.
1.30 Any of the preceding sunscreen compositions, wherein the sunscreen composition has a UVAI/UV ratio greater than or equal to 70. In certain embodiments, the sunscreen composition has a UVAI/UV ratio of about 72 or more, about 74 or more, about 76 or more, about 78 or more, about 80 or more, or about 82 or more. Additionally or alternatively, the sunscreen composition has a SPF value of 40.3 or more. For instance, the sunscreen composition may have an SPF of about 42 or more, about 45 or more, about 48 or more, about 50 or more, about 52 or more, about 55 or more, about 58 or more, about 60 or more, about 62 or more, about 65 or more, about 68 or more, about 70 or more, about 72 or more, about 75 or more, about 78 or more, or about 80 or more.

In a further aspect, the disclosure also includes a method of increasing the SPF of a sunscreen composition (e.g., Method 2.0), wherein the method comprises the addition of one or more SPF enhancement ingredients into said sunscreen composition (e.g., any of Composition 1.0 et seq.).

For example, in accordance with an aspect of the disclosure, provided is a method that includes:
2.1. Method 2.0, wherein the method comprises formulating a sunscreen composition with one or more SPF enhancement ingredients selected from: a photostabilizer (e.g., butyloctyl salicylate and/or ethylhexyl methoxycrylene), an antioxidant (e.g., a plant extract) (e.g., pongamia pinnata extract), and a combination of two or more thereof.
2.2. Method 2.0 or 2.1, wherein the SPF enhancement ingredients are selected from the group consisting of: butyloctyl salicylate, ethylhexyl methoxycrylene, pongamia pinnata extract, and combinations thereof.
2.3. Any of Methods 2.0-2.2, wherein the resulting sunscreen composition has a UVAI/UV ratio is greater than or equal to 70.
2.4. Any of the preceding methods, wherein the SPF greater than or equal to 40.
2.5. Any of the preceding methods, wherein the UVAI/UV ratio is greater than or equal to 70 and the SPF greater than or equal to 40.
2.6. Any of the preceding methods, wherein the sunscreen composition is any of Composition 1.0 et seq.

As used herein, the ultraviolet (“UV” herein) region of the electromagnetic spectrum comprises three wavebands, designated UVA from 320 to 400 nm, UVB from 290 to 320 nm and UVC from 200 to 290 nm. The UVA band can be further divided into UVAII from 315 to 340 nm and UVAI from 340 to 400 nm. The visible portion of the spectrum is generally from about 400 to about 700 nm.

As used herein, the term “active ingredient” typically refers to ingredients in the sunscreen composition that predominantly are used for absorbing UV radiation.

As used herein, the term “inactive ingredient” is used herein to mean an ingredient in the sunscreen composition that does not substantially absorb UV radiation, and may include emulsifiers, emollients, preservatives, fragrances, dyes, antioxidants, chelating agents, and similar ingredients.

As used herein, “sunscreen composition” refers to a formulation (e.g., a lotion, spray, gel or other topical product) that absorbs and/or reflects some of the sun's ultraviolet (UV) radiation and thus helps protect against negative effects of sun exposure, e.g. sunburn, premature aging, etc.

As used herein, “topically applying” or “topical application” or “topically applied” means directly spraying, wiping, laying on, or spreading on outer skin or the scalp, e.g., by use of the hands or an applicator such as a wipe, roller, or spray.

As used herein, UV radiation is divided into three bands: UVA, UVB, and UVC. All three bands are classified as probable human carcinogens. “UVA” refers to long-wavelength. UVA covers the range 315-400 nm. As used herein, UVA light wavelength is divided into UVA-I (340 nm-400 nm) and UVA-II (315 nm-340 nm).

As used herein, UVB refers to medium-wavelength. UVB covers the range 280-315 nm.

As used herein, “the pongamia pinnata seed extract” can be used interchangeably with “Pongamol” which is the chemical name for the pongamia pinnata seed extract.

In certain aspects, the composition of the disclosure, e.g., any of Composition 1.0 et seq., can comprise a cosmetically acceptable carrier. The phrase “cosmetically acceptable” means that the material is compatible with skin and hair. For example, “cosmetically acceptable carrier” means a carrier that is compatible with skin and hair, and is acceptable for application to the body.

The cosmetically acceptable carrier may include, for example, water and/or water-soluble solvents. Non-limiting examples of cosmetically acceptable carriers include glycerin, C1-4 alcohols, organic solvents, fatty alcohols, fatty ethers, fatty esters, polyols, glycols, vegetable oils, mineral oils, liposomes, laminar lipid materials, water, or any combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

In some instances, cosmetically acceptable carriers may comprise water, a mixture of water and at least one cosmetically acceptable organic solvent, or at least one cosmetically acceptable organic solvent. Additionally, cosmetically acceptable carriers may be or may include ethanol, a glycol ether, for example, dipropylene glycol n-butyl ether, isododecane, mineral oil, propylene glycol, pentylene glycol, hexylene glycol, glycerol, and mixtures thereof.

The total amount of cosmetically acceptable carrier can vary, but in some instances may be about 50 to about 95 wt. %, based on the total weight of the sunscreen composition. In some instances, the total amount of cosmetically acceptable carrier is 55 to 95 wt. %, 60 to 95 wt. %, 70 to 95 wt. %, 75 to 95 wt. %, 50 to 90 wt. %, 60 to 90 wt. %, 70 to 90 wt. %, or about 75 to about 90 wt. %, based on the total weight of the sunscreen composition.

In certain aspects, the composition of the disclosure, e.g., any of Composition 1.0 et seq., can comprise one or more organic solvents. For example, the organic solvent can be selected from any of the following: monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, glycerin, and combinations thereof. The organic solvents can be volatile or non-volatile compounds.

In some aspects the organic solvent can be selected from: ethanol, a glycol ether, for example, dipropylene glycol n-butyl ether, isododecane, mineral oil, propylene glycol, pentylene glycol, hexylene glycol, glycerol, and a mixture of two or more thereof.

In certain aspects the composition of the disclosure, e.g., any of Composition 1.0 et seq., can comprise one or more oils and/or emollients. For example, oils/emollients that may be included in the sunscreen compositions of the disclosure can be selected from any of the following: hydrocarbon-based oils of plant origin, such as liquid triglycerides of fatty acids containing from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, coriander oil, castor oil, avocado oil, caprylic/capric acid triglycerides, jojoba oil, shea butter oil and caprylyl glycol; synthetic esters and ethers of fatty acids, for instance purcellin oil, octyldodecyl neopentanoate, 2-octyldodecyl stearate, 2-octyldodecyl erucate, isostearyl isostearate; hydroxylated esters, for instance isostearyl lactate, octyl hydroxystearate, octyldodecyl hydroxystearate, glyceryl behenate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate, or isopropyl lauroyl sarcosinate; linear or branched hydrocarbons, of mineral or synthetic origin, such as volatile or non-volatile liquid paraffins, and derivatives thereof, petroleum jelly, polydecenes, isohexadecane, isododecane, hydrogenated polyisobutene such as Parleam oil, or the mixture of n-undecane (C₁₁) and of n-tridecane (C₁₃); fluoro oils that are partially hydrocarbon-based and/or silicone-based, for instance those described in document JP-A-2 295 912; silicone oils, for instance volatile or non-volatile polymethylsiloxanes (PDMS) with a linear or cyclic silicone chain, which are liquid or pasty at room temperature, in particular volatile silicone oils, especially cyclopolydimethylsiloxanes (cyclomethicones) such as cyclohexadimethylsiloxane and cyclopentadimethylsiloxane; polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, polymethylphenylsiloxanes; PEG-20 methyl glucose sesquistearate, and mixtures thereof.

In further aspects, additional specific examples of oils/emollients in the compositions of the disclosure, e.g., any of Composition 1.0 et seq., may be selected from the group consisting of: cocoglyceride, cyclomethicone, dimethicone, dicapryl maleate, caprylic/capric triglyceride, isopropyl myristate, octyl stearate, isostearyl linoleate, lanolin oil, coconut oil, cocoa butter, olive oil, avocado oil, aloe extracts, jojoba oil, castor oil, fatty acid, oleic acid, stearic acid, fatty alcohol, cetyl alcohol, hexadecyl alcohol, diisopropyl adipate, hydroxybenzoate esters, benzoic acid esters of C₉-C₁₅ alcohols, isononyl iso-nonanoate, alkanes, mineral oil, silicone, dimethyl polysiloxane, ether, polyoxypropylene butyl ether, polyoxypropylene cetyl ether, C₁₂-C₁₅ alkyl benzoate, aryl alkyl benzoate, isopropyl lauroyl sarcosinate, and any combinations thereof.

In further aspects, any of the sunscreen compositions of the disclosure (e.g., any of Composition 1.0 et seq), can include oleaginous esters, ethers, and aloe extracts, and mixtures thereof. For example, emollients that can be included in the sunscreen compositions include: Cetiol OE, Lexol IPL, octyl palmitate, isopropyl palmitate, neopentyl glycol heptanoate, neopentyl glycol diheptanoate, actiphyte of aloe vera, Trivent NP-13, and C₁₂-15 alkyl benzoate.

In some aspects, the sunscreen compositions of the disclosure (e.g., any of Composition 1.0 et seq) can include one or more conventional cosmetic oils, such as a mineral oil; hydrogenated polyisobutene; synthetic or natural-made squalane; cosmetic esters or ethers, which may be branched or unbranched, saturated or unsaturated; vegetable oils; or mixtures of two or more thereof.

Particularly suitable oils are vegetable oils with high proportions of omega-3 and omega-6 fatty acids, for example nut oils such as hazelnut oil. Other vegetable oils may also be used, such as calendula oil, jojoba oil, avocado oil, macadamia nut oil, castor oil, cocoa butter, coconut oil, corn oil, cottonseed oil, olive oil, palm kernel oil, rapeseed oil, safflower oil, sesame oil, soybean oil, sunflower seed oil, wheat germ oil, grapeseed oil, kukui nut oil, thistle oil and mixtures from that. These other oils then do not contribute so well to the desired direction of action.

The total amount of oils and/or emollients in the sunscreen composition, if present, may vary but may be about 1 to about 75 wt. %, based on the total weight of the sunscreen composition. In some instances, the total amount of oils and/or emollients may be 1 to 60 wt. %, 1 to 50 wt. %, 1 to 20 wt. %, 5 to 70 wt. %, 5 to 60 wt. %, 5 to 50 wt. %, 10 to 60 wt. %, 10 to 50 wt. %, 25 to 75 wt. %, or 30 to 60 wt. %, based on the total weight of the sunscreen composition.

In certain aspects the composition of the disclosure, (e.g., any of Composition 1.0 et seq.) can comprise one or more film-formers to ensure even coverage of UV filters and can be used to render the sunscreen composition(s) water resistant. In one aspect, the film former is a hydrophobic material that imparts film forming and/or waterproofing characteristics. For example, one such agent is a polyethylene having a molecular weight of 400. Another suitable film former is a polyethylene with a molecular weight of 2000. Yet, another suitable film former is synthetic wax. Other film-formers include any selected from the group consisting of: acrylates/acrylamide copolymer, acrylates copolymer, acrylates/C₁₂-C₂₂ alkylmethacrylate copolymer, polyethylene, waxes, VP/dimethiconylacrylate/polycarbamylpolyglycol ester, butylated PVP, PVP/hexadecene copolymer, octadecene/MA copolymer, PVP/eicosene copolymer, tricontanyl PVP, Brassica Campestris/Aleuritis Fordi Oil copolymer, decamethyl cyclopentasiloxane (and) trimethylsiloxysilicate, and mixtures thereof.

In one aspect, the film-forming polymers included in sunscreen compositions (e.g., any of Composition 1.0 et seq), are not soluble in ethanol (such as PVP/Eicosene copolymer). For example, a film-former employed in ethanol-based sunscreen products is Dermacryl LT or Dermacryl 79 marketed by Akzo Nobel (INCI Name: acrylates/octylacrylamide copolymner). Dermacryl LT (CAS Number: 80570-62-3) is a hydrophobic, high molecular weight carboxylated acrylic copolymer. It functions as a film-former in a broad range of cosmetic formulations, imparting waterproofing, increased occlusion and decreased rub-off of actives.

The total amount of film forming in the sunscreen compositions, if present, may vary but can include, in one aspect, from 0.1 to 40 wt. %, based on the total weight of the sunscreen composition. In some aspects, the total amount of film formers may be 0.1 to 30 wt. %, 0.1 to 20 wt. %, 0.1 to 10 wt. %, 1 to 40 wt. %, 1 to 30 wt. %, 1 to 20 wt. %, or 1 to 10 wt. %, based on the total weight of the sunscreen composition.

In certain aspects the composition of the disclosure, (e.g., any of Composition 1.0 et seq.) can comprise one or more emulsifier selected from the group consisting of: amphoteric, anionic, cationic or nonionic emulsifier, used alone or as a mixture, and optionally a co-emulsifier. In one aspect the emulsifiers are used when the sunscreen composition is in the form of an emulsion. For example, the emulsifiers are chosen in an appropriate manner according to the emulsion to be obtained (W/O or O/W).

For W/O emulsions, examples of emulsifiers include: hydroxyethyl acrylate/sodium acryloyldimethyl taurate copolymer, dimethicone copolyols, such as the mixture of cyclomethicone and dimethicone copolyol, and alkyl dimethicone copolyols such as lauryl dimethicone copolyol, and cetyl dimethicone copolyol. A crosslinked elastomeric solid organopolysiloxane comprising at least one oxyalkylene group, such as those obtained according to the procedure of Examples 3, 4 and 8 of U.S. Pat. No. 5,412,004 and of the examples of U.S. Pat. No. 5,811,487, for example the product of Example 3 (synthesis example) of U.S. Pat. No. 5,412,004, the contents of each of which are incorporated herein by reference, may also be used as surfactants for W/0 emulsions.

For O/W emulsions, examples of emulsifiers include any selected from the group consisting of: nonionic emulsifiers such as oxyalkylenated (e.g., polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof.

The fatty acid esters of a sugar that can be used as nonionic amphiphilic lipids can be chosen in the group consisting of esters or mixtures of esters of a C₈-C₂₂ fatty acid and of sucrose, of maltose, of glucose or of fructose, and esters or mixtures of esters of a C₁₄-C₂₂ fatty acid and of methylglucose.

The C₈-C₂₂ or C₁₄-C₂₂ fatty acids forming the fatty unit of the esters that can be used in the emulsion can comprise a saturated or unsaturated linear alkyl chain having, respectively, from 8 to 22 or from 14 to 22 carbon atoms. The fatty unit of the esters can be chosen from the group consisting of: stearates, behenates (e.g., glyceryl behenate), arachidonates, palmitates (e.g., isopropyl palmitate), myristates, laurates, caprates and mixtures thereof.

In some aspects, esters or of mixtures of esters of a fatty acid and of sucrose, of maltose, of glucose or of fructose, that can be included in any of the compositions of the disclosure, e.g., any of Composition 1.0, et seq., can be selected from the group consisting of: sucrose monostearate, sucrose distearate, sucrose tristearate and mixtures thereof, having an HLB (Hydrophilic Lipophilic Balance) of 5, 7, 11 and 16; methylglucose, disearate of methylglucose polyglycerol-3, glucose monoesters or maltose monoesters, such as methyl 0-hexadecanoyl-6-D-glucoside and O-hexadecanoyl-6-D-maltoside, and combinations thereof.

In some aspects, the fatty alcohol ethers of a sugar that can be used in the sunscreen compositions (e.g., any of Composition 1.0, et seq) as nonionic amphiphilic lipids can be chosen in from the group consisting of: ethers or mixtures of ethers of a C₈-C₂₂ fatty alcohol and of glucose, of maltose, of sucrose or of fructose, and ethers or mixtures of ethers of a C₁₄-C₂₂ fatty alcohol and of methylglucose.

In certain aspects, the C₅-C₂₂ or C₁₄-C₂₂ fatty alcohols forming the fatty unit of the ethers that can be used in the emulsion of the sunscreen compositions (e.g., any of Composition 1.0, et seq.) comprise a saturated or unsaturated linear alkyl chain having, respectively, from 8 to 22 or from 14 to 22 carbon atoms. For example, the fatty unit of the ethers can be chosen from decyl, cetyl, behenyl, arachidyl, stearyl, palmityl, myristyl, lauryl, capryl and hexadecanoyl units, and mixtures thereof such as cetearyl.

In certain aspects, the fatty alcohol ethers of a sugar, can be any selected from: alkylpolyglucosides, such as decylglucoside and laurylglucoside sold, cetostearylglucoside, optionally as a mixture with cetostearyl alcohol, and also arachidylglucoside, for example in the form of the mixture of arachidyl and behenyl alcohols and of arachidylglucoside.

In certain aspects, the sunscreen can include a nonionic amphiphilic lipid of sucrose monostearate, sucrose distearate, sucrose tristearate and mixtures thereof, the distearate of methylglucose and of polyglycerol-3, and alkylpolyglucosides. In certain aspects, the glycerol fatty esters that can be used as nonionic amphiphilic lipids can be chosen from the group consisting of: the esters formed from at least one acid comprising a saturated linear alkyl chain having from 16 to 22 carbon atoms, and from 1 to 10 glycerol units. For example, glycerol fatty esters may be used in an emulsion of the instant disclosure. For example, esters may be chosen from stearates, behenates, arachidates, palmitates, and mixtures thereof.

In other aspects, a surfactant that can be used in the emulsion of the instant disclosure (e.g., any of Composition 1.0 et seq.), can be selected from: oleth-3 phosphate, cetearyl alcohol, cetearyl glucoside, decaglycerol monostearate, distearate, tristearate and pentastearate (10 glycerol units) (CTFA names: polyglyceryl-10 stearate, polyglyceryl-10 distearate, polyglyceryl-10 tristearate, polyglyceryl-10 pentastearate), such as the products sold under the respective names Nikkol Decaglyn 1-S, 2-S, 3-S and 5-S by the company Nikko, and diglyceryl monostearate (CTFA name: polyglyceryl-2 stearate) such as the product sold by the company Nikko under the name Nikkol DGMS.

In certain further aspects, sorbitan fatty esters can be used as nonionic amphiphilic lipids, wherein the esters are selected from the group consisting: esters of a C₁₆-C₂₂ fatty acid and of sorbitan and oxyethylenated esters of a C₁₆-C₂₂ fatty acid and of sorbitan. For example, they can be formed from at least one fatty acid comprising at least one saturated linear alkyl chain, having, respectively, from 16 to 22 carbon atoms, and from sorbitol or from ethoxylated sorbitol. In one aspect, oxyethylenated esters can comprise from 1 to 100 ethylene oxide units. For example, these esters can be chosen from stearates, behenates, arachidates, palmitates and mixtures thereof. In one example, the sorbitan fatty ester and of an oxyethylenated sorbitan fatty ester, can be sorbitan monostearate (CTFA name: sorbitan stearate), sorbitan monopalmitate (CTFA name: sorbitan palmitate), or sorbitan 20 EO tristearate (CTFA name: polysorbate 65).

In one aspect, the ethoxylated fatty ethers may be ethers made up of 1 to 100 ethylene oxide units and of at least one fatty alcohol chain having from 16 to 22 carbon atoms. The fatty chain of the ethers can be chosen from behenyl, arachidyl, stearyl and cetyl units, and mixtures thereof, such as cetearyl. For example, ethoxylated fatty ethers, can include: behenyl alcohol comprising 5, 10, 20 and 30 ethylene oxide units (CTFA names: beheneth-5, beheneth-10, beheneth-20 and beheneth-30), and the ether of stearyl alcohol comprising 2 ethylene oxide units (CTFA name: steareth-2).

In one aspect, the ethoxylated fatty esters that can be used as nonionic amphiphilic lipids are esters made up of 1 to 100 ethylene oxide units and of at least one fatty acid chain comprising from 16 to 22 carbon atoms. The fatty chain of the esters can be chosen, for example, from stearate, behenate, arachidate and palmitate units, and mixtures thereof. For example, ethoxylated fatty esters can include: the ester of stearic acid comprising 40 ethylene oxide units, and the ester of behenic acid comprising 8 ethylene oxide units (CTFA name: PEG-8 behenate).

In certain further aspects, the block copolymers of ethylene oxide and of propylene oxide that can be used are chosen from poloxamers selected from: Poloxamer 231, such as the product sold by the company ICI under the name Pluronic L81 of formula (V) with x=z=6, y=39 (HLB 2); Poloxamer 282, such as the product sold by the company ICI under the name Pluronic L92 of formula (V) with x=z=10, y=47 (HLB 6); and Poloxamer 124, such as the product sold by the company ICI under the name Pluronic L44 of formula (V) with x=z=11, y=21 (HLB 16).

In further examples, any of the compositions of the disclosure (e.g., any of Composition 1.0 et seq.) can include a suitable hydrophobically-modified emulsifiers, for example, inulin lauryl carbamate.

In some aspects, any of the compositions of the disclosure, e.g., any of Composition 1.0 et seq., can include silicone-based emulsifiers (such as polysiloxanes), glycosides, polyethylene glycols, acrylic-based emulsifiers, and mixtures thereof. For example, silicone-based emulsifiers can include Abil WE 09, Abil EM-90, DC 9011 silicone elastomer, Emulgade 68/50, Arlacel P135, Simulgel A, and Simulgel EG.

In some aspects, the total amount of emulsifiers in the sunscreen compositions, if present, can be from 0.1 to 30 wt. %, based on the total weight of the sunscreen composition. In some instances, the total amount of emulsifiers is from 0.1 to 20 wt. %, 0.1 to 15 wt. %, 0.1 to 10 wt. %, 0.5 to 30 wt. %, 0.5 to 20 wt. %, 0.5 to 15 wt. %, 0.5 to 10 wt. %, 1 to 30 wt. %, 1 to 20 wt. %, 1 to 15 wt. %, 1 to 10 wt. %, based on the total weight of the sunscreen composition.

The sunscreen compositions may optionally include one or more silicone oils. Non-limiting examples of silicone oils include dimethicone, cyclomethicone, polysilicone-11, phenyl trimethicone, trimethylsilylamodimethicone, and stearoxytrimethylsilane. In some cases, the cosmetic composition includes dimethicone, and optionally additional oils, including additional silicone oils. Typically, the one or more silicone oils is a non-volatile silicon oil. In some embodiments, the silicone oil is polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl) trimethylsiloxysilicates.

Other examples of silicone oils that may be mentioned include volatile linear or cyclic silicone oils, especially those with a viscosity 8 centistokes (8×10⁶ m²/s) and especially containing from 2 to 7 silicon atoms, these silicones optionally comprising alkyl or alkoxy groups containing from 1 to 10 carbon atoms. As volatile silicone oils that may be used in the disclosure, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

The total amount of silicone oils in the sunscreen compositions, if present, can vary but is typically about 0.1 to about 40 wt. %, based on the total weight of the sunscreen composition. In some cases, the total amount of silicone oils in the sunscreen composition may be about 0.1 to about 30 wt. %, about 0.1 to about 20 wt. %, about 0.1 to about 10 wt. %, about 1 to about 40 wt. %, about 1 to about 30 wt. %, about 1 to about 20 wt. %, about 1 to about 10 wt. %, about 5 to about 40 wt. %, about 5 to about 30 wt. %, about 5 to about 20 wt. %, or about 5 to about 15 wt. %, based on the total weight of the sunscreen composition.

In some aspects, the form of the sunscreen compositions of the disclosure, e.g., any of Composition 1.0 et seq, may be in the form of a cream, a gel, a paste, a lotion, a rinse, a foam, an emulsion, a spray, etc. The sunscreen compositions may be prepared according to techniques that are well known to those skilled in the art, in particular those intended for the preparation of emulsions of oil-in-water or water-in-oil type. For example, they may be in the form of a simple or complex emulsion (O/W, W/O, O/W/O or W/O/W emulsion) such as a cream or a milk, in the form of a gel or a cream-gel, or in the form of a lotion.

In certain aspects, the sunscreen compositions of the disclosure, e.g., any of Composition 1.0 et seq, can include one or more SPF active ingredients selected from the group consisting of: Any of the preceding compositions, wherein the one or more SPF active ingredients is selected from the group consisting of: octinoxate, octisalate, homosalate, avobenzone, octocrylene, para-aminobenzoic acid, cinoxate, dioxybenzone, menthyl anthranilate, padimate O, ensulizole, sulisobenzone, trolamine salicylate, ecamsule, 4-methylbenzylidene camphor, diethylamino hydroxybenzoyl hexyl benzoate, bis diethylamino hydroxybenzoyl benzoate, disodium phenyl dibenzimidazole tetrasulfonate, Parsol SLX (INCI Polysilicone-15), Butyl Methoxydibenzoylmethane, Ethylhexyl Methoxycinnamate, Ethylhexyl Salicylate, Benzophenone-3, Ethylhexyl Dimethyl PAB A, Phenylbenzimidazole Sulfonic Acid, Benzophenone-4, Tris-biphenyl triazine, Bis-Ethylhexyloxyphenol Methoxyphenyl Triazine, Methylene Bis-Benzotriazolyl Tetramethylbutylphenol, Ethylhexyl triazone, Methoxypropylamino Cyclohexenylidene Ethoxyethylcyanoacetate, Phenylene Bis-Diphenyltriazine, Isoamyl p-Methoxycinnamate, Benzylidene Camphor Sulfonic Acid, Camphor benzalkonium methosulfate, Diethylamino Hydroxybenzoyl Hexyl Benzoate, Disodium Phenyl Dibenzimidazole Tetrasulfonate, Drometrizole Trisiloxane, PEG-25 PABA, Polyacrylamidomethyl Benzylidene Camphor, Benzophenone-5, benzophenone-8, Diethylhexyl butamido triazone, and combinations thereof.

In some aspects, either octinoxate or octisalate is included in the composition as an SPF active ingredient.

In certain aspects, the sunscreen compositions of the disclosure, e.g., any of Composition 1.0 et seq, can additional inactive ingredients, such as preservatives, fragrances, dyes, antioxidants, chelating agents, etc.

In still a further aspect, any of the sunscreen compositions of the disclosure, e.g., any of Composition 1.0 et seq, can include one or more antioxidants. For example, antioxidants that can be used in the compositions of the disclosure can be selected from the group consisting of: vitamins such as Vitamin C and derivatives thereof, for example, ascorbyl acetate, phosphate and palmitate, magnesium ascorbyl phosphate; Vitamin A and derivatives thereof; Folic acid and its derivatives, vitamin E and its derivatives, such as tocopheryl acetate; Flavones or flavonoids; Amino acids such as histidine, glycine, tyrosine, tryptophan and derivatives thereof; Imidazoles such as cis- or trans-urocanic acid and their derivatives; Peptides such as D, L-carnosine, D-carnosine, L-carnosine and their derivatives; Carotenoids and carotenes, such as α-carotene, β-carotene, lycopene; Uric acid and derivatives thereof; α-hydroxy acids such as citric acid, lactic acid, malic acid; α-hydroxy fatty acids such as palmitic acid, phytic acid, lactoferrin; Stilbenes and their derivatives; Mannose and its derivatives; Lipoic acid and its derivatives, e.g., dihydrolipoic acid; Ferulic acid and its derivatives; Thiols such as glutathione, cysteine, cystine and their esters; Folic acid and its derivatives, and combinations thereof.

In still another aspect, antioxidants or radical scavengers that can be included in any of the sunscreen compositions of the disclosure, e.g., any of Composition 1.0 et seq, are plant extracts. For example, antioxidant plant extracts can be those selected from the group consisting of Angelica archangelica, Arnica montana, Camellia sinensis, Cupressus semper, Coffee arabica, Polygonatum multiflorum, Pongamia pinnata, Rosmarinus officinalis, Evernia fufuracea, Evernia prunastri, Aventa sativa, and a mixture of two or more thereof.

In yet another aspect, any of the sunscreen compositions of the disclosure, e.g., any of Composition 1.0 et seq, comprise one or more vitamins and/or antioxidants selected from the group consisting of vitamin A (e.g., retinyl palmitate) and their derivatives, vitamin B1 (e.g., thiamine) and their derivatives, vitamin B2 (e.g., riboflavin) and their derivatives, vitamin B3 (e.g., niacin or nicotinamide/niacinamide) and their derivatives, vitamin B5 (e.g., pantothenic acid) and their derivatives, vitamin B6 (e.g., pyridoxine, pyridoxal, or pyridoxamine) and their derivatives, vitamin B12 (e.g., cyanocobalamin) and their derivatives, vitamin C (e.g., ascorbic acid) and their derivatives, vitamin E (e.g., tocopherol acetate) and their derivatives, vitamin H (e.g., biotin) and their derivatives, vitamin K (e.g., phytonadione) and their derivatives, and vitamin M (e.g., folic acid) and their derivatives, ubiquinone (also known as coenzyme Q10), glutathione, melatonin, resveratrol, citric acid, gallic acid and its esters, nordihydroguaiaretic acid, lipoic acid, dihydrolipoic acid, glycolic acid, alpha-arbutin, chitosan salicylate, rosmarinic acid, green tea extract, smithsonite extract, Angelica archangelica extract, Camellia sinensis extract, Coffee Arabica extract, Pongamia pinnata extract, and mixtures thereof.

In yet a further aspect, any of the sunscreen compositions of the disclosure, e.g., any of Composition 1.0 et seq, can include one or more photostabilizers. For example, photostabilizers that can be used in the compositions of the disclosure can be selected from the group consisting of: Tinosorb S (USAN Bemotrizinol, INCI Bis-Ethylhexyloxyphenol Methoxyphenyl Triazine); Tinosorb M (USAN Bisoctrizole, INCI Methylene Bis-Benzotriazolyl Tetramethylbutylphenol); Butyloctyl Salicylate; Hexadecyl Benzoate; Butyloctyl Benzoate; Corapan TQ (INCI Diethylhexyl 2,6-Naphthalate); Oxynex ST (INCI Diethylhexyl Syringylidene Malonate) Polycrylene (INCI Polyester-8); ethylhexyl triazone, dioctyl butamido triazone, benzylidene malonate polysiloxane, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, butyloctyl salicylate, an alpha-cyanodiphenylacrylate (e.g., ethylhexyl methoxycrylene or 2-ethylhexyl 2-cyano-3-(4-methoxyphenyl)-3-phenylpropenoate) isopentyl 4-methoxycinnamate and combinations thereof.

In yet a further aspect, any of the sunscreen compositions of the disclosure, e.g., any of Composition 1.0 et seq, can include thickening agents or gelling agents, including substances that can increase the viscosity of a composition. Thickeners include those that can increase the viscosity of a composition without substantially modifying the efficacy of the active ingredient within the sunscreen composition. Thickeners can also increase the stability of the sunscreen compositions of the present invention.

Non-limiting examples of additional thickening agents that can be used in the context of the present invention include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, 4th Ed., 1991).

Non-limiting examples of crosslinked polyacrylate polymers include cationic and nonionic polymers. Examples are described in U.S. Pat. Nos. 5,100,660; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C₁₀-C₃₀ straight chain or branched chain alkyl group through an ether linkage. For example, these can include polymers that are ethers of C₁₀-C₃₀ straight or branched chain alcohols with hydroxyalkylcelluloses.

Non-limiting examples of gums that can be used with the sunscreens of the present disclosure include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

### EXAMPLES

### Example 1

Four non-limiting, exemplary sunscreen compositions (Formulas 1-5) were prepared in accordance with aspects of the invention. The formulations of Formulas 1-5 are provided in Table 1 (below).

TABLE 1

| | Formula 1 | Formula 2 | Formula 3 | Formula 4 | Formula 5 |
|---|---|---|---|---|---|
| Ingredients | (wt. %) | (wt. %) | (wt. %) | (wt. %) | (wt. %) |
| Demineralized Water | QS to 100 | QS to 100 | QS to 100 | QS to 100 | QS to 100 |
| Preservative | 1.0% | 1.0% | 1.0% | 1.0% | 1.0% |
| Antioxidant/Vitamins | 5.1% | 5.1% | 5.1% | 5.1% | 5.1% |
| Emollients | 3.5% | 3.5% | 3.5% | 5.5% | 5.5% |
| Silicone Oil | 6.5% | 6.5% | 6.5% | 6.5% | 6.5% |
| Titanium Dioxide | 2.4% | — | — | — | 1.2% |
| Emulsifiers | 2.3% | 2.3% | 2.3% | 2.3% | 2.3% |
| Zinc Oxide | 9.4 | 11.6 | 11.6 | 11.6 | 10.4 |
| Pongamia Pinnata Seed | 0.25% | 0.25% | 0.25% | 0.25% | 0.25% |
| Extract | | | | | |
| Octisalate | 4% | — | 4% | — | 4% |
| Octinoxate | — | 3.5% | — | 2% | — |
| Ethylhexyl Methoxycrylene | 2% | 2% | 2% | 2% | 2% |
| Butyloctyl Salicylate | 2% | 1.5% | 2.0% | 2.0% | 2.0% |

### Example 2

Formulas 1-5 were evaluated to assess the SPF value, UVAI/UV ratio, and critical wavelength of such sunscreen compositions. To calculate the critical wavelength of Formulas 1-5, samples of each of Formulas 1-5 were placed in polymethylmethacrylate (PMMA) plates suitable for UV transmittance measurements. Specifically, each respective sample was applied in a series of small dots over the entire plate and then spread evenly with a gloved finger. Spreading was done with a very light spreading action for approximately 30 seconds. The plate was then allowed to equilibrate for 15 minutes in the dark prior to irradiation. The irradiation dose was equivalent to an erythemal effective dose of 800 J/m² (i.e., 80/J m²-eff).

After the irradiation, mean transmittance values were determined for each wavelength over the UV spectrum (290 to 400 nanometers) using the SPF-2905. The transmittance values were measured at 1 nanometer intervals. 5 measurements of spectral irradiation transmitted for each wavelength through a PMMA plate coated with 15 microliters of glycerin were taken and stored in the computer's memory along with the mean of the 5 measurements using the SPF-290S to obtain a reference reading. This procedure was subsequently repeated for 5 measurements with the PMMA plate coated with the test product, with the mean being computed and stored such that the computer can then ratio the mean reference reading at each wavelength with the mean test formula reading to compute the mean test formula transmittance. The mean transmittance values were then converted to mean absorbance values.

Three individual PMMA plates are utilized. Each plate is scanned five times at different locations resulting in a total of 15 measurements. The critical wavelength was identified as the wavelength at which the integral of the spectral absorbance curve reaches 90 percent of the integral over the UV spectrum from 290 to 400 nm. A mean critical wavelength of 370 nm or greater is classified as broad-spectrum protection.

The measurement of the static Sun Protection Factor (“SPF”) for Formulas 1-5 above was conducted in accordance with FDA, 21 CFR Sec. 201.327, subpart (i), SPF Test Procedure, Sunscreen Drug Products for Over-the-Counter Human Use, Final Monograph, Federal Register, Vol. 76, No. 117, Jun. 17, 2011, the contents of which are herein incorporated by reference in their entirety. Specifically, ten participants were provided with a series of five UV doses, expressed as Joules/square meter, increasing in 25% increments. This forms the basis of the unprotected MEDu. Participants were instructed to avoid additional UV exposure.

The participants were evaluated about 16 to 24 hours after completion of the MEDu doses to determine each participant's response and unprotected minimal erythemal dose (“MEDu”). The MEDu is the quantity of erythema effective energy. Subsequently, on day 2, UV doses were applied and the participant's protected MED (“MEDp”) was determined in accordance to the procedures: FDA, 21 CFR Sec. 201.327, subpart 4 (iii) and FDA, 21 CFR Sec. 201.327, subpart 5 (iii), the contents of which are incorporated herein by reference in their entireties. The participants were then evaluated about 16 to 24 hours after completion of the UV doses from Day 2. MED for all sites that received UV doses, both protected and unprotected, were evaluated. SPF values were calculated for both Formulas 1-5 based on the FDA standard using FDA, 21 CFR Sec. 201.327, subpart (i) (6), by calculating the MEDp value produced in the sunscreen protected sites to the MEDu produced in the unprotected test area, for each participant using the following calculation:

MEDip/MEDiu=SPFi value

Data from the ten participants was used for calculating the test formulations (i.e., Formulas 1-5) Label SPF Value. The mean SPF value and the Standard Deviation for the participants was also computed.

A summary of the UVAI/UV ratio, SPF value, and critical wavelength for Formulas 1-5 are provided in Table 2 (below).

TABLE 2

| | UV Filters | | | | |
|---|---|---|---|---|---|
| | (Organic UV | | | In vivo | |
| | Filters and | SPF Enhancement | UVAI/UV | SPF | Critical |
| | Metal Oxides) | Ingredients | Ratio | Value | Wavelength |
| Market Based | 7.5% Octinoxate | None | 0.63 | 46 | 370 |
| Formula | 9% Zinc Oxide | | | | |
| (Control) | | | | | |
| Formula 1 | 4% Octisalate | 2% Butyloctyl Salicylate; | 0.72 | 48 | 372.9 |
| | 2% Titanium | 2% Ethylhexyl | | | |
| | Dioxide | Methoxycrylene; | | | |
| | 9% Zinc Oxide | 0.25% Pongamia Pinnata | | | |
| | | Seed Extract | | | |
| Formula 2 | 3.5% Octinoxate | 1.5% Butyloctyl | 0.71 | 52 | 371.57 |
| | 11% Zinc Oxide | Salicylate; | | | |
| | | 2.0% Ethylhexyl | | | |
| | | Methoxycrylene; | | | |
| | | 0.25% Pongamia Pinnata | | | |
| | | Seed Extract | | | |
| Formula 3 | 4% Octisalate | 2% Butyloctyl Salicylate; | 0.77 | 45 | 372.66 |
| | 11% Zinc Oxide | 2% Ethylhexyl | | | |
| | | Methoxycrylene; | | | |
| | | 0.25% Pongamia Pinnata | | | |
| | | Seed Extract | | | |
| Formula 4 | 2% Octinoxate | 2% Butyloctyl Salicylate; | 0.75 | 46 | 372.67 |
| | 11% Zinc Oxide | 2% Ethylhexyl | | | |
| | | Methoxycrylene; | | | |
| | | 0.25% Pongamia Pinnata | | | |
| | | Seed Extract | | | |
| Formula 5 | 4% Octisalate | 2% Butyloctyl Salicylate; | 0.74 | 44 | 372.46 |
| | 1% Titanium | 2% Ethylhexyl | | | |
| | Dioxide | Methoxycrylene; | | | |
| | 10% Zinc Oxide | 0.25% Pongamia Pinnata | | | |
| | | Seed Extract | | | |

As is clear from the above table, the addition of the SPF enhancement ingredients increased the UVAI/UV ratio to above 0.7, increased the SPF to above 40, and increased the critical wavelength to above 370. The non-limiting exemplary compositions, which included certain combinations of SPF enhancement ingredients and UV filters surprisingly achieved a desirable SPF while simultaneously meeting the required UVAI/UV ratio. Moreover, Formulas 1 and 2 achieved the improvements in SPF value and UVAI/UV ratio while having a reduced total amount of UV filters as compared to the Marked Based Formula, which was unexpected.

### Example 3

The qualitative aspects of Formulas 1-5 from Example 1 were evaluated by clinical trials using eight panelists. A summary of the qualitative aspects of Formulas 1-5 and the scoring methodology are provided in Tables 3-5. For tables 3 and 4, a higher numerical value was assigned to Formulas exhibiting more of the evaluated feature and a lower numerical value was assigned to Formulas exhibiting less of the evaluated feature. The viscosities in Table 4 were measured using a viscometer.

TABLE 3

| Product | | | | | |
|---|---|---|---|---|---|
| Appearance | Formula 1 | Formula 2 | Formula 3 | Formula 4 | Formula 5 |
| Integrity of | 7/8 | 7/8 | 5/8 | 6/8 | 4/8 |
| Shape | | | | | |
| Gloss | 2/8 | 2/8 | 2/8 | 1/8 | 4/8 |

TABLE 4

| | Formula 1 | Formula 2 | Formula 3 | Formula 4 | Formula 5 |
|---|---|---|---|---|---|
| Wetness | 1/8 | 3/8 | 4/8 | 6/8 | 3/8 |
| Spreadability | 0/8 | 2/8 | 6/8 | 7/8 | 3/8 |
| Absorbance | 1/8 | 2/8 | 6/8 | 7/8 | 2/8 |
| Thickness | 8/8 | 7/8 | 0/8 | 1/8 | 6/8 |
| Whiteness | 7/8 | 6/8 | 0/8 | 0/8 | 6/8 |
| Stickiness | 5/8 | 1/8 | 2/8 | 0/8 | 2/8 |
| Amount of | 7/8 | 4/8 | 0/8 | 2/8 | 5/8 |
| Residue | | | | | |
| Slip | 3/8 | 3/8 | 7/8 | 3/8 | 4/8 |
| Preference | 2/8 | 3/8 | 8/8 | 6/8 | 3/8 |
| Viscosity | 19100 | 15100 | 13900 | 15900 | 17400 |
| (cP) | | | | | |

TABLE 5
Scoring

| Number of times attribute is selected | Summary |
|---|---|
| 0-1 | Statistically Significant |
| 2-6 | Not Statistically Significant |
| 7-8 | Statistically Significant |

The above scoring is based on the selection of the formulation relative to the Market Based Formula described in Example 2. For example, eight panelists (out of eight) preferred Formula 3 relative to the Market Based Formula, and six panelists (out of eight) preferred Formula 4 relative to the Market Based Formula.

### Example 4

Five non-limiting exemplary sunscreen compositions (Formulas 6-10) were prepared in accordance with aspects of the disclosure. The formulation for such sunscreen compositions is provided in Table 6 (below).

TABLE 6

| | Formula 6 | Formula 7 | Formula 8 | Formula 9 | Formula 10 |
|---|---|---|---|---|---|
| | (wt. %) | (wt. %) | (wt. %) | (wt. %) | (wt. %) |
| Deionized Water | QS to 100 | QS to 100 | QS to 100 | QS to 100 | QS to 100 |
| Polyether-1 | 0.18 | 0.18 | 0.18 | 0.18 | 0.18 |
| Citric Acid | 0.01 | 0.01 | 0.01 | 0.01 | 0.01 |
| Preservatives | 1 | 1 | 1 | 1 | 1 |
| (Hydroxyacetophenone,1,2- | | | | | |
| hexanediol (and) caprylyl | | | | | |
| glycol, and/or | | | | | |
| Phenoxyethanol (and) | | | | | |
| Ethylhexylglycerin) | | | | | |
| Dimethicone | 1 | 1 | 1 | 1 | 1 |
| Petrolatum | 7 | 7 | 7 | 7 | 7 |
| Emollient | 5.5 | 5.3 | 5.3 | 5.3 | 7.3 |
| Pongamia Pinnnata Seed | 0.25 | 0.25 | 0.25 | 0.25 | 0.25 |
| Extract | | | | | |
| Octisalate | 5 | 5 | 5 | | |
| Octinoxate | | | | 3.5 | 3.5 |
| Ethylhexyl Methoxycrylene | 2 | 2 | 2 | 2 | 2 |
| Butyloctyl Salicylate | 2 | 2 | 2 | 2 | 1.5 |
| Emulsifier | 2.45 | 2.65 | 0.85 | 0.85 | 2.65 |
| Zinc Oxide (and) | 11.1 | 11.1 | 11.1 | 11.1 | 11.1 |
| Triethoxycaprylsilane | | | | | |
| Glyceryl Behenate | | | 0.5-1 | 0.5-1 | |
| Carnosine | | 0.05-0.3 | 0.05-0.3 | 0.05-0.3 | 0.05-0.3 |
| Bisabolol and Ginger Extract | | 0.05-0.3 | 0.05-0.3 | 0.05-0.3 | 0.05-0.3 |
| Sodium Hyaluronate | | 0.001-0.01 | 0.001-0.01 | 0.001-0.01 | 0.001-0.01 |
| Vitamin E (e.g., d-gamma | 0.005-0.1 | 0.1-0.5 | 0.1-0.5 | 0.1-0.5 | 0.1-0.5 |
| Tocopherol) | | | | | |

### Example 5

Five non-limiting exemplary sunscreen compositions (Formulas 11-15) were prepared in accordance with aspects of the disclosure. The formulation for such sunscreen compositions is provided in Table 7 (below).

TABLE 7

| | Formula | Formula | Formula | Formula | Formula |
|---|---|---|---|---|---|
| | 11 | 12 | 13 | 14 | 15 |
| Ingredients | (wt. %) | (wt. %) | (wt. %) | (wt. %) | (wt. %) |
| Demineralized Water | QS to 100 | QS to 100 | QS to 100 | QS to 100 | QS to 100 |
| Preservative | 1 | 1 | 1 | 1 | 1 |
| Antioxidant/Vitamins | 5.1 | 5.1 | 5.1 | 5.1 | 5.1 |
| Emollients | 3.5 | 3.5 | 3.5 | 5.5 | 5.5 |
| Silicone Oil | 6.5 | 6.5 | 6.5 | 6.5 | 6.5 |
| Titanium Dioxide | 2.4 | | | | 1.2 |
| Emulsifiers | 2.3 | 2.3 | 2.3 | 2.3 | 2.3 |
| Zinc Oxide (and) | 9.4 | 11.6 | 11.6 | 11.6 | 10.4 |
| Triethoxycaprylysilane | | | | | |
| Pongamia Pinnata Seed | 0.25 | 0.25 | 0.25 | 0.25 | 0.25 |
| Extract | | | | | |
| Octisalate | 4 | | 4 | | 4 |
| Octinoxate | | 3.5 | | 2 | |
| Ethylhexyl | 2 | 2 | 2 | 2 | 2 |
| Methoxycrylene | | | | | |
| Butyloctyl Salicylate | 2 | 1.5 | 2 | 2 | 2 |
| Carnosine | 0.05-0.3 | 0.05-0.3 | 0.05-0.3 | 0.05-0.3 | 0.05-0.3 |
| Bisabolol and Ginger | 0.05-0.3 | 0.05-0.3 | 0.05-0.3 | 0.05-0.3 | 0.05-0.3 |
| Extract | | | | | |
| Sodium Hyaluronate | 0.001-0.01 | 0.001-0.01 | 0.001-0.01 | 0.001-0.01 | 0.001-0.01 |
| Vitamin E (e.g., d-gamma | 0.05-0.3 | 0.05-0.3 | 0.05-0.3 | 0.05-0.3 | 0.05-0.3 |
| Tocopherol) | | | | | |

### Example 6

Seven compositions (Formulas 16-22) were prepared in accordance with aspects of the invention. Formulas 16-22 were evaluated according to the procedures described in Example 2 to determine the SPF value and UVAI/UV ratio. The change in SPF value relative to Formula 16 (ΔSPF_{X-Formula 16}), which was employed as a control, was calculated. A summary of the formulations for Formulas 16-22 as well as the SPF value and the UVAI/UV ratio for each respective composition is provided in Table 8 (below).

TABLE 8

| | Formula | Formula | Formula | Formula | Formula | Formula | Formula |
|---|---|---|---|---|---|---|---|
| | 16 | 17 | 18 | 19 | 20 | 21 | 22 |
| Water (wt. %) | Q.S to | Q.S to | Q.S to | Q.S to | Q.S to | Q.S to | Q.S to |
| | 100 | 100 | 100 | 100 | 100 | 100 | 100 |
| Lactic Acid (wt. %) | 0.005-0.1 | 0.005-0.1 | 0.005-0.1 | 0.005-0.1 | 0.005-0.1 | 0.005-0.1 | 0.005-0.1 |
| Niacinamide (wt. %) | 5 | 5 | 5 | 5 | 5 | 5 | 5 |
| Preservative | 1 | 1 | 1 | 1 | 1 | 1 | 1 |
| Dimethicone (wt. %) | 6.5 | 6.5 | 6.5 | 6.5 | 6.5 | 6.5 | 6.5 |
| Tocopheryl Acetate | 0.1 | 0.1 | 0.1 | 0.1 | 0.1 | 0.1 | 0.1 |
| (wt. %) | | | | | | | |
| Octyldodecyl | 3 | 3 | 3 | 3 | 3 | 3 | 3 |
| Neopentanoate (wt. %) | | | | | | | |
| Emulsifiers | 2.4 | 2.4 | 2.4 | 2.4 | 2.4 | 2.4 | 2.4 |
| (Hydroxyethyl | | | | | | | |
| Acrylate/Sodium | | | | | | | |
| Acryloyldimethyl | | | | | | | |
| Taurate Copolymer | | | | | | | |
| and/or Oleth-3 | | | | | | | |
| Phosphate) (wt. %) | | | | | | | |
| Octinoxate (wt. %) | 3.5 | 3.5 | 3.5 | 3.5 | 3.5 | 3.5 | 3.5 |
| Zinc Oxide (and) | 11.6 | 11.6 | 11.6 | 11.6 | 11.6 | 11.6 | 11.6 |
| triethoxthycaprylylsilane | | | | | | | |
| (wt. %) | | | | | | | |
| Butyloctyl Salicylate | | 1.5 | | | | 1.5 | 1.5 |
| (wt. %) | | | | | | | |
| Ethylhexyl | | | 2 | | 2 | 2 | 2 |
| Methoxycrylene (wt. %) | | | | | | | |
| Pongamia Extract | | | | 0.25 | 0.25 | | 0.25 |
| (wt. %) | | | | | | | |
| ΔSPF % _{X-Formula 16} (%) | — | −10.6 | −4 | 1.4 | −3.8 | −9.8 | 19.9 |
| ΔSPF_{X-Formula 16} | — | −2.01 | −0.76 | +0.27 | −0.71 | −1.85 | +3.77 |
| UVAI/UV ratio | 0.743 | 0.737 | 0.75 | 0.733 | 0.72 | 0.734 | 0.764 |
| Critical Wavelength | 369 | 369 | 370 | 369 | 369.6 | 370 | 371 |

As seen in Table 8, certain combinations of SPF active ingredients, metal oxides, photo stabilizers, and/or antioxidants achieved significant improvements in the UVAI/UV ratio while simultaneously achieving a desirable SPF Value. Moreover, the significant improvement in SPF value achieved by Formula 22 while simultaneously having an improvement in the UVAI/UV ratio was unexpected in view of Formula 17, which indicates that adding butyloctyl salicylate to octinoxate and zinc oxide reduces the SPF value and the UVAI/UV ratio of the sunscreen composition; in view of Formula 20, which indicates that adding ethylhexyl methoxycrylene and pongamia extract to octinoxate and zinc oxide reduces the SPF value and the UVAI/UV ratio of the sunscreen composition; and in view of Formula 21, which indicates that adding butyloctyl salicylate and ethylhexyl methoxycrylene to octinoxate and zinc oxide reduces the SPF value and the UVAI/UV ratio of the sunscreen composition. Formula 22 was further evaluated by way of in-vivo testing and determined to have a SPF value of 52.

Formula 22 and the Market Based Formula from Example 1 were evaluated by an expert panel to assess the whiteness of such formulas. Formula 22 exhibited a similar whiteness to the Market Based Formula, which was unexpected as Formula 22 contained about 22% more zinc oxide than the Market Based Formula.

### Example 7

Eight compositions (Formulas 23-30) were prepared in accordance with aspects of the invention. Formulas 23-30 were evaluated according to the procedures described in Example 2 to determine the SPF value and UVAI/UV ratio. A summary of the formulations for Formulas 23-30 as well as the SPF value and the UVAI/UV ratio for each respective composition is provided in Table 9.

TABLE 9

| | Formula | Formula | Formula | Formula | Formula | Formula | Formula | Formula |
|---|---|---|---|---|---|---|---|---|
| | 23 | 24 | 25 | 26 | 27 | 28 | 29 | 30 |
| Water (wt. %) | Q.S. to | Q.S. to | Q.S. to | Q.S. to | Q.S. to | Q.S. to | Q.S. to | Q.S. to |
| | 100 | 100 | 100 | 100 | 100 | 100 | 100 | 100 |
| Polyether-1 (wt. %) | 0.18 | 0.18 | 0.18 | 0.18 | 0.18 | 0.18 | 0.18 | 0.18 |
| Phenoxyethanol | 1 | 1 | 1 | 1 | 1 | 1 | 1 | 1 |
| (and) | | | | | | | | |
| Ethylhexylglycerin | | | | | | | | |
| (wt. %) | | | | | | | | |
| Dimethicone | 1 | 1 | 1 | 1 | 1 | 1 | 1 | 1 |
| (wt. %) | | | | | | | | |
| Petrolatum (wt. %) | 7 | 7 | 7 | 7 | 7 | 7 | 7 | 7 |
| Emulsifiers (PEG- | 1.8 | 1.8 | 1.8 | 1.8 | 1.8 | 1.8 | 1.8 | 1.8 |
| 20 Methyl Glucose | | | | | | | | |
| Sesquistearate, | | | | | | | | |
| Cetearyl Alcohol | | | | | | | | |
| & Cetearyl | | | | | | | | |
| Glucoside, and | | | | | | | | |
| Oleth-3 | | | | | | | | |
| Phosphate) | | | | | | | | |
| Isopropyl | 5 | 5 | 5 | 5 | 5 | 5 | 5 | 5 |
| Palmitate (wt. %) | | | | | | | | |
| Tocopheryl | 0.15 | 0.15 | 0.15 | 0.15 | 0.15 | 0.15 | 0.15 | 0.15 |
| Acetate (wt. %) | | | | | | | | |
| Bisabolol & | 0.01-0.5 | 0.01-0.5 | 0.01-0.5 | 0.01-0.5 | 0.01-0.5 | 0.01-0.5 | 0.01-0.5 | 0.01-0.5 |
| Ginger Extract | | | | | | | | |
| (wt. %) | | | | | | | | |
| Carnosine (wt. %) | 0.01-0.5 | 0.01-0.5 | 0.01-0.5 | 0.01-0.5 | 0.01-0.5 | 0.01-0.5 | 0.01-0.5 | 0.01-0.5 |
| d-gamma | 0.01-0.5 | 0.01-0.5 | 0.01-0.5 | 0.01-0.5 | 0.01-0.5 | 0.01-0.5 | 0.01-0.5 | 0.01-0.5 |
| tocopherol (wt. %) | | | | | | | | |
| Citric Acid (wt. %) | 0.001-0.1 | 0.001-0.1 | 0.001-0.1 | 0.001-0.1 | 0.001-0.1 | 0.001-0.1 | 0.001-0.1 | 0.001-0.1 |
| Sodium | 0.001-0.1 | 0.001-0.1 | 0.001-0.1 | 0.001-0.1 | 0.001-0.1 | 0.001-0.1 | 0.001-0.1 | 0.001-0.1 |
| Hyaluronate | | | | | | | | |
| (wt. %) | | | | | | | | |
| Hydroxyethyl | 1.2 | 1.2 | 1.2 | 1.2 | 1.2 | 1.2 | 1.2 | 1.2 |
| Acrylate/Sodium | | | | | | | | |
| Acryloyldimethyl | | | | | | | | |
| Taurate | | | | | | | | |
| Copolymer (wt. %) | | | | | | | | |
| Octisalate (wt. %) | 5 | 5 | 5 | 5 | 5 | 5 | 5 | 5 |
| Zinc Oxide (and) | 11 | 11 | 11 | 11 | 11 | 11 | 11 | 11 |
| triethoxylcaprylyl- | | | | | | | | |
| silane (wt. %) | | | | | | | | |
| Butyloctyl | | 2 | | | | 2 | 2 | 2 |
| Salicylate (wt. %) | | | | | | | | |
| Ethylhexyl | | | 2 | | 2 | 2 | | 2 |
| Methoxycrylene | | | | | | | | |
| (wt. %) | | | | | | | | |
| Pongamia Extract | | | | 0.25 | 0.25 | | 0.25 | 0.25 |
| (wt. %) | | | | | | | | |
| ΔSPF % _{X-Formula 23} | — | 8.19 | 50.0 | 5.21 | 77.30 | 33.13 | 4.22 | 79.9 |
| (%) | | | | | | | | |
| ΔSPF_{X-Formula 23} | — | 0.66 | 4.03 | 0.42 | 6.23 | 2.67 | 0.34 | 6.44 |
| UVAI/UV ratio | 0.79 | 0.76 | 0.766 | 0.787 | 0.793 | 0.769 | 0.762 | 0.772 |
| Critical | 370 | 370 | 371 | 370 | 371.4 | 371.4 | 370 | 371 |
| Wavelength | | | | | | | | |

As seen in Table 9 (above), certain combinations of SPF active ingredients, metal oxides, photo stabilizers, and/or antioxidants achieved significant improvements in the UVAI/UV ratio while simultaneously achieving a desirable SPF Value. The significant improvement in SPF Value and UVAI/UV ratio achieved by certain sunscreen compositions disclosed in Table 9 (e.g., but not limited to, Formulas 27 and 30) was unexpected and surprising.

Formula 30 and the Market Based Formula from Example 1 were evaluated by an expert panel to assess the whiteness of such formulas. Formula 30 exhibited a similar whiteness to the Market Based Formula, which was unexpected as Formula 30 contained about 22% more zinc oxide than the Market Based Formula. Formula 30 was evaluate pursuant to in-vivo testing and was determined to have an SPF of 51.

### Example 8

A composition (Formula 31) was prepared in accordance with aspects of the invention. Formula 31 was evaluated according to the procedures described in Example 2 to determine the SPF value and UVAI/UV ratio. A summary of the formulation for Formula 31 as well as the SPF value and the UVAI/UV ratio for Formula 31 is provided in Table 10.

TABLE 10

| | Formula 31 |
|---|---|
| Water (wt. %) | Q.S. to 100 |
| Lactic Acid (wt. %) | 0.001-0.1 |
| Niacinamide (wt. %) | 5 |
| Hydroxyacetophenone (wt. %) | 0.5 |
| 1,2-Hexanediol (and) Caprylyl Glycol | 0.5 |
| (wt. %) | |
| Sodium Hyaluronate (wt. %) | 0.001-0.1 |
| Octyldodecyl Neopentanoate (wt. %) | 5 |
| Dimethicone (wt. %) | 5.5 |
| Oleth-3 Phosphate (wt. %) | 0.001-0.1 |
| Tocopheryl Acetate (wt. %) | 0.15 |
| Bisabolol (and) Zingiber Officinale | 0.05-0.4 |
| (Ginger) Root Extract (wt. %) | |
| d-gamma tocopherol (wt. %) | 0.05-0.4 |
| Hydroxyethyl Acrylate/Sodium | 2.1 |
| Acryloyldimethyl Taurate Copolymer | |
| (wt. %) | |
| ZnO (wt. %) | 11 |
| Butyloctyl Salicylate (wt. %) | 5 |
| Ethylhexyl Methoxycrylene (wt. %) | 2.5 |
| Pongamia Pinnata Seed Extract (wt. %) | 0.06 |
| SPF value | 45 |
| UVAI/UV ratio | 0.77 |
| Critical Wavelength | 371 |

### Example 9

A composition (Formula 32) was prepared in accordance with aspects of the invention. The formulation of Formula 32 is provided in Table 11.

TABLE 11

| | Formula 32 |
|---|---|
| Water (wt. %) | Q.S. to 100 |
| Polyether-1 (wt. %) | 0.18 |
| Phenoxyethanol (and) | 1 |
| Ethylhexylglycerin (wt. %) | |
| Dimethicone (wt. %) | 1 |
| Petrolatum (wt. %) | 7 |
| Emulsifiers (PEG-20 Methyl | 3.3 |
| Glucose Sesquistearate, Cetearyl | |
| Alcohol & Cetearyl Glucoside, | |
| and Oleth-3 Phosphate) | |
| Isopropyl Palmitate (wt. %) | 5 |
| Tocopheryl Acetate (wt. %) | 0.15 |
| Bisabolol & Ginger Extract | 0.01-0.5 |
| (wt. %) | |
| Carnosine (wt. %) | 0.01-0.5 |
| d-gamma tocopherol (wt. %) | 0.01-0.5 |
| Citric Acid (wt. %) | 0.001-0.1 |
| Sodium Hyaluronate (wt. %) | 0.001-0.1 |
| Hydroxyethyl Acrylate/Sodium | 1.2 |
| Acryloyldimethyl Taurate | |
| Copolymer (wt. %) | |
| Octisalate (wt. %) | 5 |
| Zinc Oxide (wt. %) | 11 |
| Butyloctyl Salicylate (wt. %) | 2 |
| Ethylhexyl Methoxycrylene | 2 |
| (wt. %) | |
| Pongamia Extract (wt. %) | 0.25 |

Formula 32 was evaluated in comparison to the Market Based Formula from Example 1 by an expert panel to assess the whiteness of Formula 32. The expert panel applied a sample of the respective composition and evaluated the witnesses of the respective sample after three rubs during application, after twelve rubs during application, at end of application of the sample, the immediate whiteness, and whiteness after five minutes. Table 12 includes a summary of the evaluation of the Formula 32 and the Market Based Formula.

TABLE 12

| | Market Based | |
|---|---|---|
| | Formula | Formula 32 |
| Whiteness at 3 rubs | 23.6 | 18.9 |
| Whiteness at 12 rubs | 15 | 11.4 |
| Whiteness at end of | 4.8 | 4 |
| application | | |
| Whiteness - Immediate | 3 | 2.5 |
| Whiteness at 5 mins | 1.2 | 1.4 |

As seen in Table 12, Formula 32 exhibited less whiteness than the Market Based Formula. This was unexpected as Formula 32 had about 22% more zinc oxide than the Marked Based Formula.

The invention has been described above with reference to illustrative Examples, but it is to be understood that the invention is not limited to the disclosed embodiments. Alterations and modifications that would occur to one of skill in the art upon reading the specification are also within the scope of the invention, which is defined in the appended claims.

1. A sunscreen composition comprising:
a metal oxide in an amount from 5 to 25 wt. %, relative to the total weight of the sunscreen composition;
one or more SPF active ingredient in an amount from about 1 to about 10 wt. %; and
one or more SPF enhancement component in an amount from about 0.1 to about 10 wt. %, the one or more SPF enhancement component comprising a photostabilizer, an antioxidant, or a combination thereof,
wherein the UVAI/UV ratio of the sunscreen is greater than or equal to 70 and the SPF greater than or equal to 40.

2. (canceled)

3. The sunscreen composition of claim 1, wherein the metal oxide comprises zinc oxide, titanium oxide, or a combination thereof.

4. (canceled)

5. The sunscreen composition of claim 1, wherein the one or more SPF active ingredient is selected from octinoxate, octisalate, homosalate, avobenzone, para-aminobenzoic acid, cinoxate, dioxybenzone, menthyl anthranilate, octocrylene, padimate O, ensulizole, sulisobenzone, trolamine salicylate, ecamsule, 4-methylbenzylidene camphor, diethylamino hydroxybenzoyl hexyl benzoate, bis diethylamino hydroxybenzoyl benzoate, disodium phenyl dibenzimidazole tetrasulfonate, Parsol SLX (INCI Polysilicone-15), Butyl Methoxydibenzoylmethane, Ethylhexyl Methoxycinnamate, Ethylhexyl Salicylate, Benzophenone-3, Ethylhexyl Dimethyl PABA, Phenylbenzimidazole Sulfonic Acid, Benzophenone-4, Tris-biphenyl triazine, Bis-Ethylhexyloxyphenol Methoxyphenyl Triazine, Methylene Bis-Benzotriazolyl Tetramethylbutylphenol, Ethylhexyl triazone, Methoxypropylamino Cyclohexenylidene Ethoxyethylcyanoacetate, Phenylene Bis-Diphenyltriazine, Isoamyl p-Methoxycinnamate, Benzylidene Camphor Sulfonic Acid, Camphor benzalkonium methosulfate, Diethylamino Hydroxybenzoyl Hexyl Benzoate, Disodium Phenyl Dibenzimidazole Tetrasulfonate, Drometrizole Trisiloxane, PEG-25 PABA, Polyacrylamidomethyl Benzylidene Camphor, Benzophenone-5, benzophenone-8, Diethylhexyl butamido triazone, and a combination of two or more thereof.

6. The sunscreen composition of claim 1, wherein the SPF active ingredient comprises octinoxate, octisalate, or a combination thereof.

7-9. (canceled)

10. The sunscreen composition of claim 1, wherein the SPF enhancement ingredient comprises an antioxidant selected from Angelica archangelica, Arnica montana, Camellia sinensis, Cupressus sewer, Coffee arabica, Polygonatum multiflorum, Pongamia pinnata extract, Rosmarinus officinalis, Evernia fufuracea, Evernia prunastri, Aventa sativa, vitamin A, vitamin E, peptides, flavones, flavonols, and combinations thereof.

11-14. (canceled)

15. The sunscreen composition of claim 1, wherein the photostabilizer is selected from ethylhexyl triazone, dioctyl butamido triazone, benzylidene malonate polysiloxane, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, butyloctyl salicylate, an alpha-cyanodiphenylacrylate (e.g., ethylhexyl methoxycrylene or 2-ethylhexyl 2-cyano-3-(4-methoxyphenyl)-3-phenylpropenoate), isopentyl 4-methoxycinnamate, and combinations thereof.

16. The sunscreen composition of claim 1, wherein the sunscreen composition comprises:
zinc oxide;
octinoxate, octisalate, or a combination thereof; and
butyloctyl salicylate, ethylhexyl methoxycrylene, pongamia pinnata extract).

17. The sunscreen composition of claim 1, wherein the sunscreen composition comprises:
from about 5 to about 15 wt. % of zinc oxide;
from about 1 to about 10 wt. % of octisalate;
from about 0.5 to about 5 wt. % of butyloctyl salicylate;
from about 0.5 to about 5 wt. % of ethylhexyl methoxycrylene; and
from about 0.1 to 1 wt. % of pongamia pinnata extract,
wherein all weight percentages are relative to the total weight of the sunscreen composition.

18. (canceled)

19. The sunscreen composition of claim 1, wherein the sunscreen composition comprises:
from about 5 to about 15 wt. % of zinc oxide;
from about 1 to about 10 wt. % of octinoxate;
from about 0.5 to about 5 wt. % of butyloctyl salicylate;
from about 0.5 to about 5 wt. % of ethylhexyl methoxycrylene; and
from about 0.1 to 1 wt. % of pongamia pinnata extract,
wherein all weight percentages are relative to the total weight of the sunscreen composition.

20-25. (canceled)

26. A sunscreen composition comprising:
a SPF enhancement component comprising pongamia pinnata extract, ethylhexyl methoxycrylene, butyloctyl salicylate, or a combination of two or more thereof;
an organic UV filter; and
a metal oxide.

27. The sunscreen composition according to claim 26, wherein the SPF enhancement component comprises two or more of pongamia pinnata extract, ethylhexyl methoxycrylene, or butyloctyl salicylate.

28. The sunscreen composition according to claim 26, wherein the SPF enhancement component comprises pongamia pinnata extract and ethylhexyl methoxycrylene.

29. The sunscreen composition according to claim 26, wherein the SPF enhancement component comprises pongamia pinnata extract, ethylhexyl methoxycrylene, and butyloctyl salicylate.

30. The sunscreen composition according to claim 26, wherein the organic UV filter is octisalate.

31. The sunscreen composition according to claim 29, wherein the organic UV filter is octinoxate.

32. The sunscreen composition according to claim 26, wherein the SPF enhancement component consists of pongamia pinnata extract and the organic UV filter is octinoxate.

33. The sunscreen composition according to claim 26, wherein the organic UV filter is selected from octinoxate, octisalate, homosalate, avobenzone, para-aminobenzoic acid, cinoxate, dioxybenzone, menthyl anthranilate, octocrylene, padimate O, ensulizole, sulisobenzone, trolamine salicylate, ecamsule, 4-methylbenzylidene camphor, diethylamino hydroxybenzoyl hexyl benzoate, bis diethylamino hydroxybenzoyl benzoate, disodium phenyl dibenzimidazole tetrasulfonate, Parsol SLX (INCI Polysilicone-15), Butyl Methoxydibenzoylmethane, Ethylhexyl Methoxycinnamate, Ethylhexyl Salicylate, Benzophenone-3, Ethylhexyl Dimethyl PAB A, Phenylbenzimidazole Sulfonic Acid, Benzophenone-4, Tris-biphenyl triazine, Bis-Ethylhexyloxyphenol Methoxyphenyl Triazine, Methylene Bis-Benzotriazolyl Tetramethylbutylphenol, Ethylhexyl triazone, Methoxypropylamino Cyclohexenylidene Ethoxyethylcyanoacetate, Phenylene Bis-Diphenyltriazine, Isoamyl p-Methoxycinnamate, Benzylidene Camphor Sulfonic Acid, Camphor benzalkonium methosulfate, Diethylamino Hydroxybenzoyl Hexyl Benzoate, Disodium Phenyl Dibenzimidazole Tetrasulfonate, Drometrizole Trisiloxane, PEG-25 PABA, Polyacrylamidomethyl Benzylidene Camphor, Benzophenone-5, benzophenone-8, Diethylhexyl butamido triazone, and a combination of two or more thereof.

34. The sunscreen composition according to claim 26, wherein the metal oxide comprises titanium oxide, iron oxide, zinc oxide, zirconium oxide, cerium oxide, or a combination of two or more thereof.

35. The sunscreen composition according to claim 26, wherein the metal oxide comprises zinc oxide, titanium oxide, or a combination thereof.

37. The sunscreen composition according to claim 26, wherein the sunscreen composition has a UVAI/UV ratio greater than or equal to 70 and the sunscreen composition has a SPF value greater than or equal to 40.3.

38. (canceled)
